(19) United States
(12) Patent Application Publication
(10) Pub. No.: US 2022/0369674 A1
(43) Pub. Date: Nov. 24, 2022

(54) VIBRATING SHAKER TRAY DISINFECTANT DEVICE THAT USES ULTRAVIOLET LIGHT

(71) Applicant: Steribin, LLC, St. George, UT (US)

(72) Inventors: Wayne Provost, Ivins, UT (US); Jonathan M. Cole, St. George, UT (US); Jeffrey Mel Glauser, Santa Clara, UT (US)

(73) Assignee: Steribin, LLC, St. George, UT (US)

(21) Appl. No.: 17/745,569

(22) Filed: May 16, 2022

Related U.S. Application Data
(60) Provisional application No. 63/190,329, filed on May 19, 2021.

(51) Int. Cl. A23L 3/00; A61L 2/10; A23L 3/28; A23L 3/015

(52) U.S. Cl. CPC A23L 3/001; A61L 2/10; A23L 3/28; A23L 3/015

(57) ABSTRACT

A disinfectant device provides a solution to disinfect bulk quantities of small items such as food items. The disinfectant device has a housing, an inlet, an outlet, a shaker tray positioned to descend from a first height on the inlet side to a second height on the outlet side, the second height lower than the first height, wherein the shaker tray is configured to shake or vibrate via a motor, and at least one disinfecting light is positioned within the housing and above the shaker tray, the light configured to disinfect items passing on the shaker tray. The disinfectant device may have a hopper having a gate to the shaker tray. The gate may be manually operated or actuated by a motor to control the number of items on the shaker tray at any given time, thereby ensuring adequate exposure to the disinfecting light.

### CROSS-REFERENCE TO RELATED APPLICATIONS

This application claims priority to U.S. Provisional Application Ser. No. 63/190,329, filed on May 19, 2021, which is incorporated herein by reference.

### TECHNICAL FIELD

The present disclosure relates to a device to disinfect items. More particularly, the present disclosure relates to a device to disinfect bulk quantities of small items.

### BACKGROUND

Infectious diseases commonly spread through the direct transfer of bacteria, viruses, or other microbes from contact with contaminated surfaces. Accordingly, disinfecting surfaces of items is important to maintaining health throughout the world. When surfaces are not disinfected to remove the viruses and bacteria thereon, people may become ill. Disinfection of small items in bulk, such as fruit, nuts, olives, or other items is accomplished in the prior art via washing, spraying with chemicals, air scrubbing, vacuuming, brushing, and other methods. However, despite the efforts of the prior art, bacteria and other contaminants remain. Additionally, chemical sprays and soaps may not be suitable for eating if not thoroughly washed, posing a hazard to consumers. Accordingly, there is a need for a disinfectant device that can thoroughly disinfect bulk quantities of small items without using chemicals or soaps. The present disclosure seeks to solve these and other problems.

### SUMMARY OF EXAMPLE EMBODIMENTS

In some embodiments, a disinfectant device comprises a housing, an inlet, an outlet, a shaker tray positioned to descend from a first height on the inlet side to a second height on the outlet side, the second height lower than the first height, wherein the shaker tray is configured to shake or vibrate via motor, and at least one disinfecting light is positioned within the housing and above the shaker tray, the light configured to disinfect items passing on the shaker tray. In some embodiments, the disinfectant device comprises one or more doors/gates for controlling the number of items passing through the inlet. In some embodiments, the disinfectant device comprises one or more fans.

In some embodiments, the inlet comprises a hopper having a gate to the shaker tray. The gate may be actuated by a motor and a controller. In some embodiments, the controller uses one or more sensors to detect items in the hopper and to detect items on the shaker tray.

In one method of use, bulk items (e.g., nuts, berries) are fed into the inlet where they drop into the hopper leading to the shaker tray. A gate is positioned at the bottom of the hopper, controlling access to the shaker tray. The gate may be manually actuated (e.g., hand-maneuvered rod) or controlled by a motor and a controller.

### BRIEF DESCRIPTION OF THE DRAWINGS

FIG. 1 illustrates a top, side perspective view of a disinfectant device;

FIG. 2 illustrates a side, longitudinal cross-section of a disinfectant device;

FIG. 3 illustrates a front elevation of a disinfectant device;

FIG. 4 illustrates a rear elevation view of a disinfectant device;

FIG. 5 illustrates a top plan view of a disinfectant device;

FIG. 6 illustrates a side, longitudinal cross-section of a disinfectant device; and

FIG. 7 illustrates a side, longitudinal cross-section of a disinfectant device.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.). While ultraviolet (UV) light is used as an example throughout, it will be appreciated that any light, or electromagnetic wavelength of light, capable of destroying or inhibiting the growth of microorganisms is contemplated herein as a “disinfecting light.”

As previously discussed, there is a need for a disinfectant device that can thoroughly disinfect bulk quantities of small items without using chemicals or soaps. The disinfectant device disclosed herein seeks to solve these and other problems.

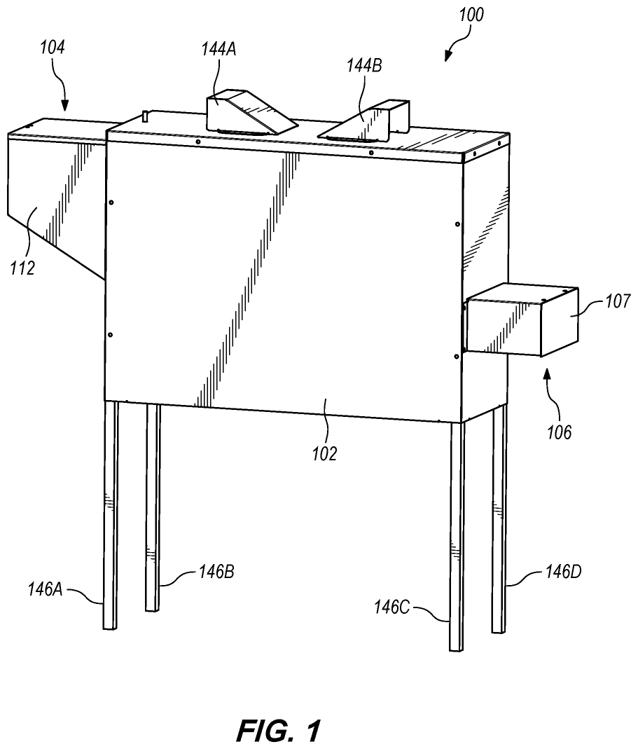

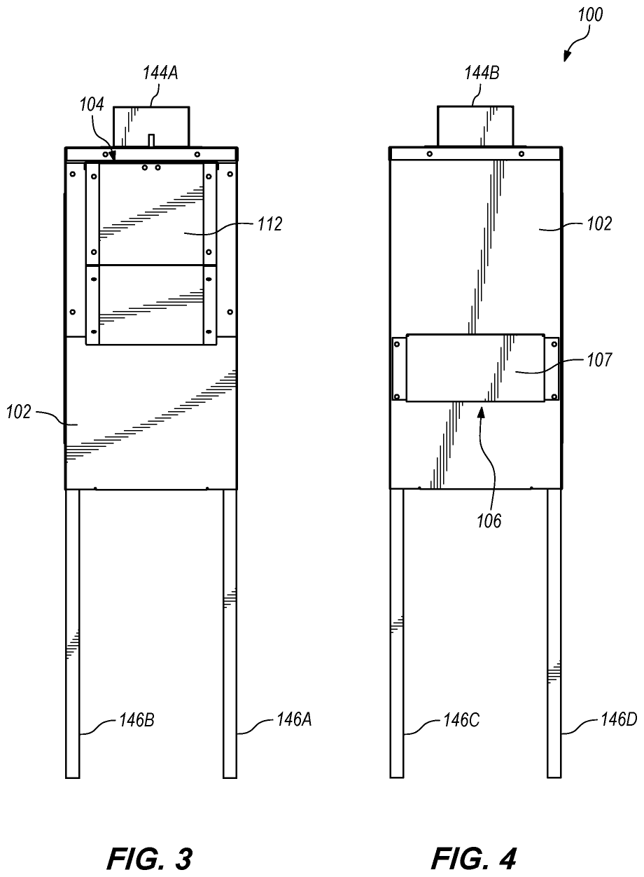

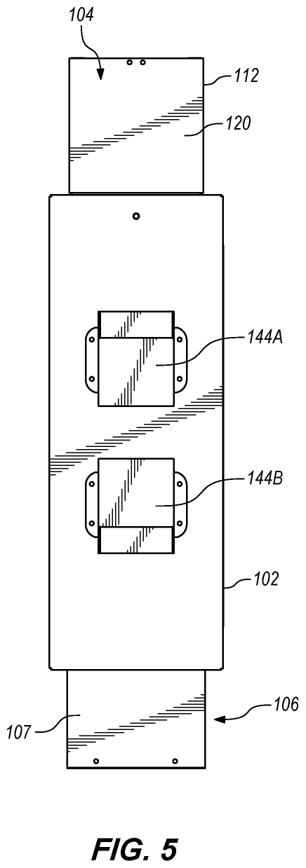

In some embodiments, as shown in FIGS. 1-5, a disinfectant device 100 comprises a housing 102, an inlet 104, an outlet 106, a shaker tray 108, an at least one disinfecting light 110. The inlet 104 comprises a hopper 112 for holding bulk items that are in need of disinfecting. A gate 114 separates the hopper 112 from an interior 116 of the housing 102. In some embodiments, the gate 114 is manually adjustable by a user, such as by withdrawing or inserting a gate rod 118. For example, when the gate rod 118 is in a fully inserted position in the housing 102, the gate 114 prevents items from passing from the hopper 112 to the interior 116. A user may control the number of items that may pass from the hopper 112 to the interior 116, and therefore the shaker tray 108, by withdrawing the gate rod 118 from the housing 102 to a desired position. In other words, at least a portion of the gate rod 118 is accessible from outside the housing 102 where it may be actuated by a user. For example, the gate rod 118 may be threaded so as to allow for minor adjustments. A user may withdraw or insert the gate rod 118 to the desired position so that the desired flow of items from the hopper 112 to the shaker tray 108 is achieved. The gate 114 may also be controlled via a motor, as discussed later herein.

The hopper 112 may further comprise an inlet door 120, which may be hinged or removable, to prevent unwanted items from falling into the hopper 112. If the gate 114 is open, items pass from the hopper 112 to the shaker tray 108. As shown, the shaker 108 has a first height at a first end 122 and a second, lower height, at a second end 124. In other words, the shaker tray 108 descends from the side with the inlet 104 to the outlet 106. As a result, the items may be gravity fed from the hopper 112 down the shaker tray 108 and out the outlet 106. To ensure that the items are distributed and disinfected on all surfaces as they travel down the shaker tray 108, the shaker tray 108 comprises one or more vibrating and/or shaker motors 126. To allow for movement, the shaker tray 108 may be supported via one or more springs 127A-B or shock absorbers 129A-B or some combination. In other words, as the motor 126 vibrates/shakes the shaker tray 108, the springs 127A-B and/or shock absorbers 129A-B allow the shaker tray 108 to shake and/or vibrate while absorbing/dampening the resulting movement.

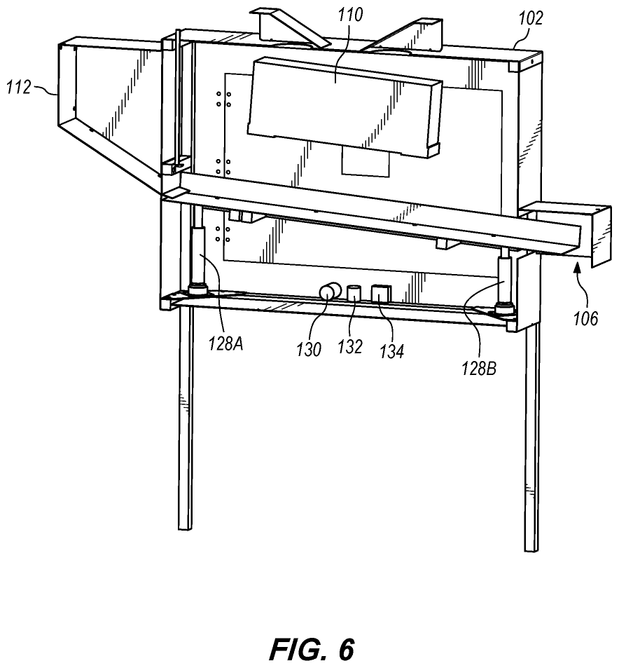

In some embodiments, as shown in FIG. 6, the shaker tray 108 may be supported by one or more linear actuators 128A-B. In some embodiments, the linear actuators 128A-B are hydraulic and are coupled to a motorized pump 130 and hydraulic reservoir 132 that controls the linear actuators 128A-B. The motorized pump 130 may be configured to vary the hydraulic pressure, thereby causing the linear actuators 128A-B to alternate rapidly, thereby shaking the shaker tray 108. In some embodiments, a controller (e.g., microcontroller) 134 may be used to control the motorized pump 130. It will be appreciated that the linear actuators 128-B need not by hydraulic, but can also be electric and driven electrically by one or more motors.

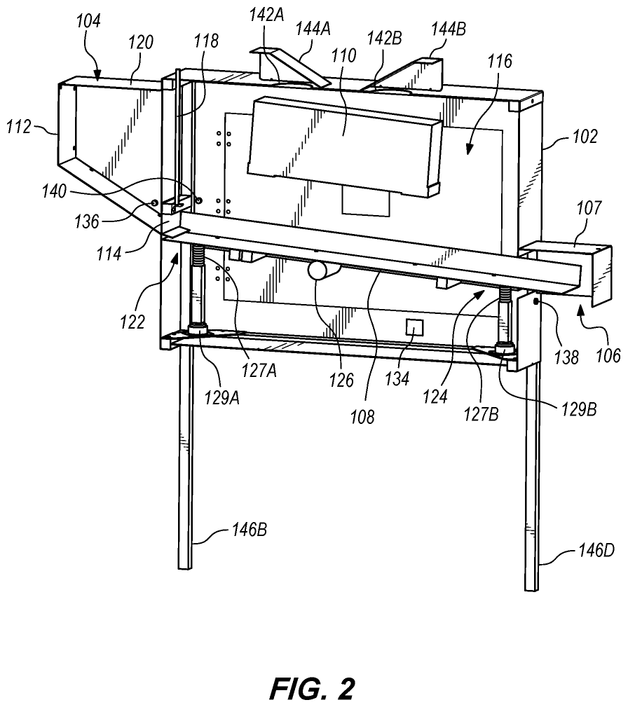

Returning back to FIG. 2, a controller 134 may also be used control the vibrating/shaking motor 126 that is coupled to the shaker tray 128. Further, the controller 134 may receive signals from one or more sensors. For example, in some embodiments, a hopper sensor 136 detects the presence of items within the hopper 112. For example, if the controller 134 determines, via the hopper sensor 136, that there are no items in the hopper 112, the controller may turn off the motor 126. Further, an outlet sensor 138 may be positioned near the outlet 106 to detect the speed or number of items exiting the outlet 106. The controller 134 may then adjust the speed of the motor 126 to either increase or decrease the flow of items as they pass down the shaker tray 108 to the outlet 106. In some embodiments, the gate 114 may comprise a switch 140 for turning on/off the motor 126 and/or controller 134. For example, when the gate 114 is in a closed position, the switch 140 (or alternatively, a sensor) is in a first position, cutting power to the motor 126. When the gate 114 is in a lifted, or open, position, the switch 140 is in a second position, providing power to the motor 126 and/or controller 134. In other words, if the gate 114 is closed and no items are entering the shaker tray 108, there is no need to shake or vibrate the shaker tray 108. Once the gate 114 is opened, the motor 126 may turn on to shake or vibrate the shaker tray 108. In some embodiments, even when the gate 114 is open, the controller 134 receives signals from the hopper sensor 136 and/or outlet sensor 138 to control the power status of the motor 126.

In some embodiments, the shaker tray 108 may exit the interior 116 of the housing 102 at the outlet 106. The outlet 106 may comprise a shield 107 to protect items as they exit the outlet 106 and to direct the items into a receiver or onto a conveyor belt, as desired by a user. Further, in some embodiments, the housing 102 may comprise one or more fans 142A-B to allow airflow in the interior 116 of the housing 102. The fans may have hoods 144A-B to prevent unwanted items from coming into contact with the fans 142A-B, respectively. Further, the housing 102 may be supported legs 146A-D.

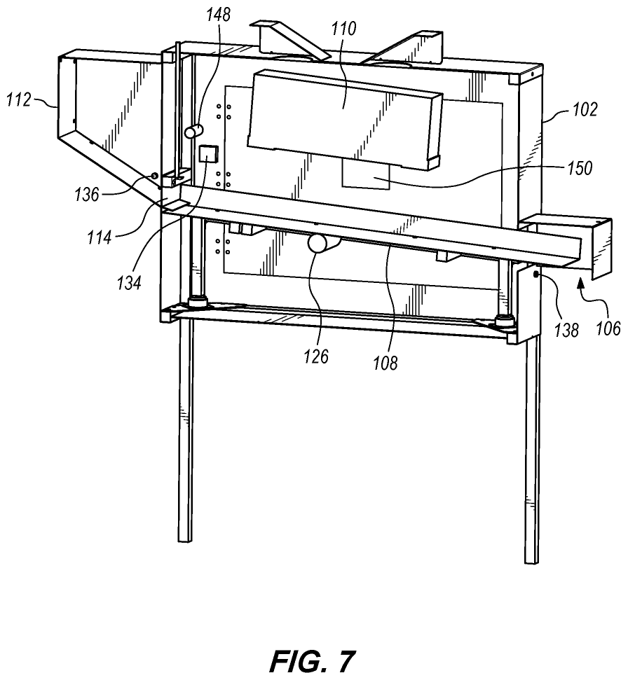

In some embodiments, as shown in FIG. 7, the gate 114 may be controlled via a gate motor 148. Further, the gate motor 148 may operable via a controller 134. For example, the controller 134 may determine, via hopper sensor 136, whether there are items in the hopper 112. If items are detected, the controller initiates the gate motor 148 to raise the gate 114, thereby opening it and allowing items to flow from the hopper 112 to the first end 122 of the shaker tray 108. Additionally, the controller 134 (or a separate controller) may initiate the vibrating/shaking motor 126 so that items may be distributed and gravity fed down the shaker tray 108 to the second end 124 and to the outlet 106. The gate motor 148 and the vibrating/shaking motor 126 may be adjusted by the controller 134 in response to signals from the outlet sensor 138. In other words, if the controller determines, via the outlet sensor 138, that items are exiting too rapidly or in too great of quantities, the controller may reduce the opening of the gate 114 and/or slow the frequency of the vibrating/shaking motor 126. In some embodiments, the housing 102 may comprise a viewing window 150 so that a user can view the items on the shaker tray 108 so as to ensure even distribution and exposure to the disinfecting light 108. The viewing window 150 may have a cover to prevent light from escaping when not being used, and may also have tint or other film thereon for ease of viewing therethrough. The sensors disclosed herein may be of any suitable type, including infrared, laser, mechanical switches, or others.

Accordingly, in one method of use, bulk items (e.g., nuts, fruits, or other items, including non-food items) are fed into the inlet 104 where they drop into the hopper 112 leading to the shaker tray 108. A gate 114 is positioned at the bottom of the hopper 112, controlling the flow of items to the shaker tray 108. The gate 114 may be manually actuated or electrically controlled. As items enter the interior of the housing 116 and onto the shaker tray 108, a shaking/vibrating motor 126 shakes/vibrates the shaker tray 108 so that the items are gravity fed from the inlet 104 to the outlet 106. By shaking/vibrating, the items are distributed along the shaker tray 108 where they are exposed to disinfecting light 110. Further, the shaking/vibrating causes the items to alternate surfaces on the shaker tray 108, thereby ensuring that all surfaces of the items are exposed to the disinfecting light 110 and thereby disinfected. Once disinfected, the items exit the outlet 106. It will be appreciated that the pitch of the of the shaker tray 108 from the first end 122 to the second end 124 may be varied to change the speed with which items are gravity fed, in addition to changing gate 114 height and intensity of the shaking/vibrating motor 126.

As a result, the disinfecting device disclosed herein overcomes shortcoming in the prior art by thoroughly sanitizing small items in bulk without harsh chemicals or other treatments.

Further, although generally referred to herein as a “disinfecting device,” it is understood that a disinfecting device of the present disclosure may disinfect, sterilize, sanitize, or otherwise treat and clean the surface of a contaminated item to achieve a lessened state or condition of contamination. Housing 102 may include an exterior material having a first property or function, and an interior material having a second property or function that is different than the first property or function. For example, in at least one embodiment, the housing 102 may include an exterior material that is structurally rigid and opaque, and an interior material that is reflective. In some embodiments, the interior material of the housing 102 may include a coating applied to an inner surface of the exterior material of the housing 102.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A disinfectant device, comprising:
a housing having an inlet and an outlet, the inlet comprising a hopper;
a gate positioned between the hopper and an interior of the housing, the gate operable to open or close access to the interior from the hopper;
a shaker tray comprising a first end having a first height and a second end having a second height, wherein the second height is lower than the first height;
a motor configured to shake or vibrate the shaker tray; and
at least one disinfecting light positioned in the interior of the housing above the shaker tray.

2. The disinfectant device of claim 1, further comprising a gate motor configured to control the gate.

3. The disinfectant device of claim 2, further comprising a hopper sensor and a controller configured to detect the presence of items within the hopper, the controller configured to control the position of the gate based upon input received from the hopper sensor.

4. The disinfectant device of claim 1, further comprising an outlet sensor and a controller configured to detect the speed or amount of items exiting the outlet.

5. The disinfectant device of claim 1, further comprising shock absorbers configured to support the shaker tray.

6. The disinfectant device of claim 1, further comprising an inlet door to the hopper.

7. The disinfectant device of claim 1, further comprising at least one fan.

8. The disinfectant device of claim 1, wherein the second end of the shaker tray extends out of the interior of the housing.

9. The disinfectant device of claim 8, wherein the outlet comprises a shield positioned over the second end of the shaker tray.

10. A disinfectant device, comprising:
a housing having an inlet and an outlet, the inlet comprising a hopper;
a gate positioned between the hopper and an interior of the housing, the gate operable to open or close access to the interior from the hopper;
a gate motor configured to actuate the gate;
a shaker tray comprising a first end having a first height and a second end having a second height, wherein the second height is lower than the first height;
a motor configured to shake or vibrate the shaker tray;
at least one disinfecting light positioned in the interior of the housing above the shaker tray;
at least one controller configured to control the gate motor and the motor of the shaker tray; and
at least one sensor for providing signals to the at least one controller.

11. The disinfectant device of claim 10, wherein the at least one sensor comprises a hopper sensor.

12. The disinfectant device of claim 10, wherein the at least one sensor comprises a sensor configured to detect a position of the gate.

13. The disinfectant device of claim 10, wherein the at least one sensor comprises an outlet sensor configured to detect the speed or amount of items as the items exit the outlet.

14. The disinfectant device of claim 10, wherein the shaker tray is supported by one or more shock absorbers.

15. The disinfectant device of claim 10, further comprising an inlet door to the hopper.

16. The disinfectant device of claim 10, further comprising at least one fan.

17. The disinfectant device of claim 10, wherein the second end of the shaker tray extends out of the interior of the housing.

18. The disinfectant device of claim 17, wherein the outlet comprises a shield positioned over the second end of the shaker tray.

19. A method of disinfecting items using the disinfectant device, the method comprising:
feeding a plurality of items into a hopper of the disinfecting device;
actuating the gate so that the plurality of items are fed onto a shaker tray at a user determined rate;
using a shaking or vibrating motor to shake and gravity feed the plurality of items from a first end of the shaker tray to a second end;
using a disinfecting light positioned above the shaker tray to disinfect the plurality of items as they pass thereunder on the shaker tray; and
the plurality of items exiting through an outlet.
